(19) United States
(12) Patent Application Publication
(10) Pub. No.: US 2008/0096963 A1
(43) Pub. Date: Apr. 24, 2008

(54) Use Of A Fatty Acid For Preparing A Topical Composition For Allaying Inflammatory Reaction Due To Vennemous Hymenoptera Strings

(75) Inventors: Theirry Jean, Paris (FR); Fabrice Balavoine, Paris (FR); Mark Crawford, Ne Bothell, WA (US)

Correspondence Address: DORSEY & WHITNEY LLP;INTELLECTUAL PROPERTY DEPARTMENT, 250 PARK AVENUE, NEW YORK, NY, 10177, US

(73) Assignee: CEREP

(21) Appl. No.: 11/572,437

(22) PCT Filed: Jul. 18, 2005
(86) PCT No.: PCT/FR05/01826

§ 371 Date: Jan. 22, 2007

(30) Foreign Application Priority Data
Jul. 21, 2004 (FR) 0408070

(51) Int. Cl. A61K 31/20; A61K 31/201; A61K 31/202; A61P 29/00

(52) U.S. Cl. 514/560; 514/558

(57) ABSTRACT

The invention relates to the use of a fatty acid for preparing a composition for allaying inflammatory reactions due to hypenoptera stings.

The present invention relates to the use of a fatty acid for the preparation of a topical composition for allaying inflammatory reactions due to hymenoptera stings.

Insects that secrete substances harmful to humans are rather numerous in our part of the world. Between wasps, hornets, bumblebees, ants and honeybees, the risks of being stung are not insignificant. These insects are called hymenoptera. Their sting can cause local or generalized non-allergic reactions as well as local or generalized allergic reactions.

Local non-allergic reactions are due to a nonspecific inflammatory reaction. A typical reaction to a hymenoptera sting consists of immediate pain combined with redness, itching and edema in an area several centimeters in diameter. The progression of the reaction is variable. The reaction can last a few hours (three on average) and generally does not last more than one day. Particular types of reactions do exist, however. Stings that lead to non-allergic reactions at specific sites can have dramatic consequences. Stings in the oral cavity, in particular at the back of the throat, can lead to respiratory disorders due to local edema. Finally, stings to the cornea of the eye can cause immediate complications such as an ocular abscess or perforation of the globe. Certain complications may appear later, including cataracts or glaucoma.

Generalized non-allergic reactions may be encountered when multiple stings cause severe envenomation. These reactions include digestive disorders with diarrhea and vomiting, a drop in blood pressure occasionally associated with palpitations, sporadic convulsions and an attack on the muscles that can lead to renal failure (in the context of rhabdomyolysis). These reactions generally appear after more than approximately thirty stings.

Allergic reactions concern roughly 1% of the population. They can be local, regional, generalized or delayed. They are generally categorized according to severity: local reaction (broader than the normal reaction but limited to a limb), regional reaction (reaction that reaches the joints of the stung limb), generalized reaction (distal skin, respiratory symptoms, Quincke's edema, asthma, digestive symptoms, palpitations), and anaphylactic shock (drop in blood pressure in addition to the symptoms described, spontaneously fatal).

The hymenoptera include a number of species including bees, wasps, hornets and ants. Although most hymenoptera stings cause only moderate pain and a limited, temporary reaction, they may be dangerous, even fatal, if multiple stings occur, if they occur in the mouth, in the throat, or to the eye, or if the stung subject has an allergic reaction. The stung subject initially feels a more or less sharp pain depending on the type of insect and the quantity of venom injected. The skin around the sting becomes red and swollen. The subject feels itching that is more or less intense.

Recent work has shown that insect venom, in particular bee venom, is a complex substance comprised of a large number of active compounds including histamine, melittin, hyaluronidase and phospholipase A. Sensitivity to these venom proteins may explain some of the reactions described above.

The applicant thus has found, surprisingly, that these fatty acids, in particular C12-C24 acids, considerably reduce the inflammatory reaction that follows a hymenoptera sting.

Thus, the present invention relates to the use of a fatty acid for preparing a topical or injectable composition for allaying inflammatory reactions due to hymenoptera stings.

The fatty acids used in the present invention are preferably natural fatty acids, i.e., capable of being obtained from natural product such as oils, but also synthetic fatty acids that are identical to or are different from natural fatty acids.

Moreover, suitable fatty acids may be saturated or unsaturated fatty acids. The salts or the pharmaceutically acceptable derivatives of these fatty acids that are also fatty acids may also be used.

Among the fatty acids used within the framework of the present invention, the following may be cited in particular: C12 to C24 acids, lauric (n-dodecanoic) acid, myristic (n-tetradecanoic) acid, palmitic (n-hexadecanoic) acid, stearic (n-octadecanoic) acid, arachidic (n-eicosanoic) acid, behenic (n-docosanoic) acid, lignoceric (n-tetracosanoic) acid, palmitoleic (cis-Δ⁹-hexadecenoic) acid, oleic (cis-Δ⁹-octadecenoic) acid, linoleic (cis,cis-Δ⁹-,Δ¹²-octadecadienoic) acid, linolenic (all-cis-Δ⁹-,Δ¹²-,Δ¹⁵-octadecatrienoic) acid, and arachidonic (all-cis-Δ⁵-,Δ⁸-,Δ¹¹-,Δ¹⁴-eicosatetraenoic) acid.

Polyunsaturated fatty acids are preferred, and linoleic acid and oleic acid are particularly active in the use according to the invention.

The fatty acids were tested in vitro for their capacity to inhibit the activity of certain pro-inflammatory components of bee venom, and tested in vivo in rat in models of bee venom induced edemas.

The results of these tests show that fatty acids can be used to produce compositions for local treatment of inflammation resulting from venomous hymenoptera stings.

In the present case, the composition to which the invention relates may also include other active ingredients, notably an anesthetic and/or an antibiotic and/or an anti-allergic or anti-inflammatory substance.

The fatty acids can be formulated in any form suitable for topical administration, in combination with suitable excipients, to allow administration of a dose of 0.01 mg to 50 mg per venomous hymenoptera sting. Of course, the dose may vary according to the quantity of venom absorbed, the number of stings or the type of insect.

Among the galenical formulations which may be suitable for implementing the invention, ointments, creams, gels, patches, powders, sprays and lotions may be cited. Application via a stick may prove particularly advantageous for a single sting on a limb, for example. If the mucosa or the eyes are stung, a formulation in the form of a spray or a collyrium, respectively, is preferred.

In certain cases it will be preferable to administer the composition according to the present invention via local injection. The active substances of the pharmaceutical compositions according to the invention may be dissolved or suspended in a pharmaceutically-acceptable sterile injectable liquid, such as sterile water, sterile organic solvent or a mixture of these two liquids for local administration in a dose of 0.001 mg to 1 mg per venomous hymenoptera sting.

The invention will be better understood upon consideration of the examples below which refer to the following figures:

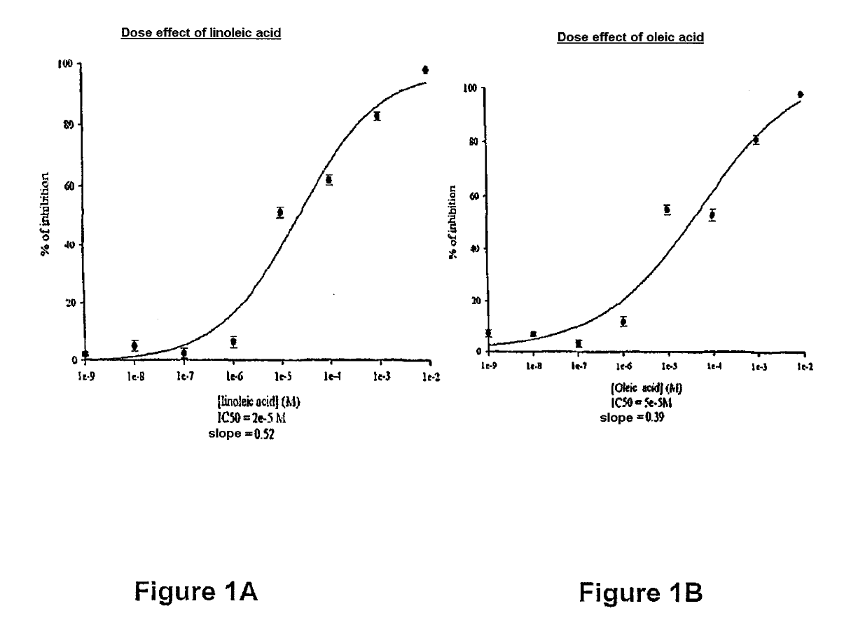

FIG. 1: Percentage of inhibition of bee venom PLA2 activity as a function of acid concentration as follows:

FIG. 1A: Linoleic acid (example 1)

FIG. 1B: Oleic acid (example 1)

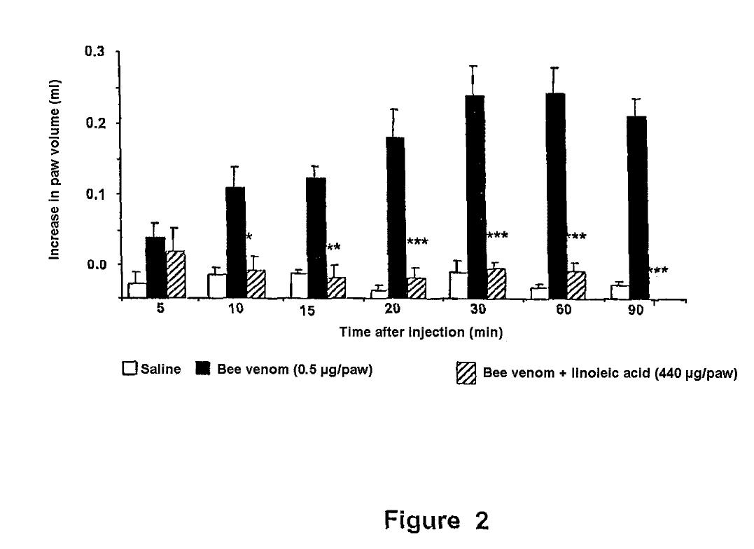

FIG. 2: Effect of oleic acid and linoleic acid on rat-paw volume increase in an inflammation model as a function of time following bee venom injection (example 2, experiment 1).

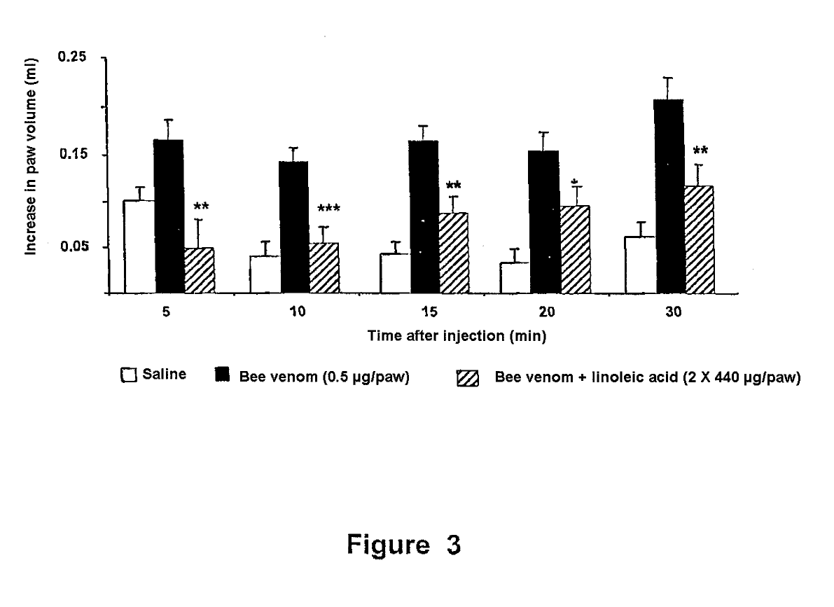

FIG. 3: Effect of oleic acid and linoleic acid on rat-paw volume increase in an inflammation model as a function of time following bee venom injection (example 2, experiment 2).

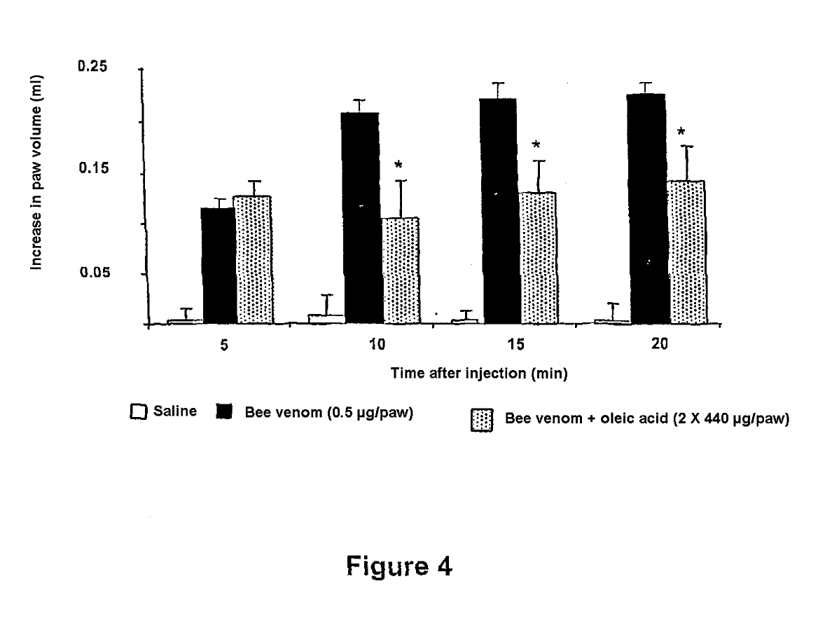

FIG. 4: Effect of oleic acid and linoleic acid on rat-paw volume increase in an inflammation model as a function of time following bee venom injection (example 2, experiment 3).

### EXAMPLE I

### In vitro inhibiting effects of linoleic acid and oleic acid on bee venom phospholipase A2 (PLA2) activity, a major inflammatory component of said venom.

The effect of linoleic acid and oleic acid on PLA2 activity is measured according to the hexane extraction method described by M. Katsumata, G. Gupta, and A. S. Goldman, (1986), Anal. Biochem. 154 (2), 676-681. PLA2 activity is determined by using a radioactive substrate, L-a-dipalmitoyl-[2,9,10(N)-3H-palmitoyl]-phosphatidylcholine. The reaction is carried out in 1 ml of glycine/NaOH buffer, pH 9, containing 2.2 mM deoxycholate, 0.11 μCi of dipalmitoyl-PC and 32 mU/ml of bee venom PLA2. After 20 minutes of incubation, the reaction is quenched by adding 0.2 ml of a Triton X-100/EDTA solution, and then the reaction product, radiolabeled palmitic acid, is extracted by a hexane solution containing 0.1% acetic acid and 0.7 g/ml Na₂SO₄. The radioactivity of the extract is determined using a liquid scintillation counter. The results represent the mean±SEM of the CPM values obtained in two independent experiments.

FIGS. 1A and 1B illustrate the percentage of inhibition of PLA2 activity (vertical axis) as a function of the concentration of the fatty acid used (horizontal axis).

These results show that linoleic acid and oleic acid inhibit the enzymatic activity of bee venom PLA2 in a dose-dependent manner.

### EXAMPLE II

Activity of oleic acid and linoleic acid in a rat model of inflammatory edema caused by bee venom (G. A. Rabinovtch, E. C. Sotomayor, C. M. Riera, I. Bianco and S. G. Correa (2000), Eur. J. Immunol. 30, 1331-1339; J. Chen, C. Luo, H. L. Li, and H. S. Chen, (1999), Pain, 83, 67-76).

Experiment 1:

Bee venom (20 μg/ml in 0.9% NaCl) is incubated at room temperature in the presence or absence of linoleic acid (32 μM). Thirty minutes after incubation begins, the solutions (25 μl) are injected subcutaneously in the upper surface of the foot of Sprague-Dawley rats (110-140 g). Edema is measured using a plethysmometer at 5, 10, 15, 20, 30, 60 and 90 minutes following injection. The results represent the mean±SEM of 5 animals. Statistical comparisons are performed using a t-test, for which the control group is comprised of animals having received venom alone. (*p<0.05, ***p<0.005) The results are represented in FIG. 2.

Experiment 2:

At 120 and 30 minutes before the injection of bee venom (20 μ0.9% NaCl), the Sprague-Dawley rats (110-140 g) either receive or do not receive, on the top of the foot, an application of topical linoleic acid. Edema is measured using a plethysmometer at 5, 10, 15, 20 and 30 minutes following subcutaneous injection of bee venom in the upper surface of the animal's foot. The results (FIG. 3) represent the mean+SEM of 20 animals. Statistical comparisons are performed using a t-test, for which the control group is comprised of animals treated with the carrier (acetone).(*p<0.05, **p<0.01, ***p<0.005)

Experiment 3:

At 120 and 30 minutes before the injection of bee venom (20 μg/ml in 0.9% NaCl), the Sprague-Dawley rats (110-140 g) either receive or do not receive, on the top of the foot, an application of topical oleic acid. Edema is measured using a plethysmometer at 5, 10, 15 and 20 minutes following subcutaneous injection of bee venom in the upper surface of the animal's foot. The results (FIG. 4) represent the mean±SEM of 5 animals. Statistical comparisons are performed using a t-test, for which the control group is comprised of animals treated with the carrier (acetone). (*p<0.05)

1-6. (canceled)

7. A method for allaying an inflammatory reaction due to a hymenoptera sting, comprising:
administering a composition comprising a fatty acid to a subject,
wherein the composition is administered at least one of topically or by injection.

8. The method according to claim 7, wherein the fatty acid comprises a C12-C24 acid.

9. The method according to claim 7, wherein the fatty acid comprises at least one of lauric (n-dodecanoic) acid, myristic (n-tetradecanoic) acid, palmitic (n-hexadecanoic) acid, stearic (n-octadecanoic) acid, arachidic (n-eicosanoic) acid, behenic (n-docosanoic) acid, lignoceric (n-tetracosanoic) acid, palmitoleic (cis-⁹-hexadecenoic) acid, oleic (cis-Δ⁹-octadecenoic) acid, linoleic (cis,cis-Δ⁹-,Δ¹²-octadecadienoic) acid, linolenic (all-cis-Δ⁹-,Δ¹²-,Δ¹⁵-octadecatrienoic) acid, or arachidonic (all-cis-Δ⁵-,Δ⁸-,Δ¹¹-,Δ¹⁴-eicosatetraenoic) acid.

10. The method according to claim 7, wherein the fatty acid is selected from the group consisting of lauric (n-dodecanoic) acid, myristic (n-tetradecanoic) acid, palmitic (n-hexadecanoic) acid, stearic (n-octadecanoic) acid, arachidic (n-eicosanoic) acid, behenic (n-docosanoic) acid, lignoceric (n-tetracosanoic) acid, palmitoleic (cis-Δ⁹-hexadecenoic) acid, oleic (cis-Δ⁹-octadecenoic) acid, linoleic (cis,cis-Δ⁹-,Δ¹²-octadecadienoic) acid, linolenic (all-cis-Δ⁹-,Δ¹²-, Δ¹⁵-octadecatrienoic) acid, and arachidonic (all-cis-Δ⁵-,Δ⁸ ,Δ¹¹-,Δ¹⁴-eicosatetraenoic) acid.

11. The method according to claim 7, wherein the fatty acid comprises at least one of linoleic acid or oleic acid.

12. The method according to claim 7, wherein the fatty acid is selected from the group consisting of linoleic acid and oleic acid.

13. The method according to claim 7, wherein the composition has a form of at least one of an ointment, a cream, a gel, a patch, a powder, a spray, a lotion or a stick.

14. The method according to claim 7, wherein the composition is provided in an injectable form.

15. The method according to claim 7, wherein the composition is applied to a region of tissue close to the sting.
